(19) United States
(12) Patent Application Publication
(10) Pub. No.: US 2018/0028071 A1
(43) Pub. Date: Feb. 1, 2018

(54) Wearable patch for measuring temperature and electrical signals

(71) Applicant: VivaLnk Inc., Santa Clara, CA (US)

(72) Inventor: Wei Shi, San Jose, CA (US)

(21) Appl. No.: 15/414,549

(22) Filed: Jan. 24, 2017

Related U.S. Application Data
(63) Continuation-in-part of application No. 15/224,121, filed on Jul. 29, 2016.

(51) Int. Cl. A61B 5/01; A61B 5/00

(52) U.S. Cl. CPC A61B 5/01; A61B 5/0008; A61B 5/6814; A61B 5/6833

(57) ABSTRACT

A wearable patch includes a stretchable and permeable substrate having a first opening, a temperature sensing unit mounted in the stretchable and permeable substrate, wherein the temperature sensing unit comprises a temperature sensor for measuring a user's skin temperature, electrodes attached to the stretchable and permeable substrate. The electrodes include a bottom surface that can contact with the user's skin and to pick up electric signals from the user's body, and a circuit substrate on the stretchable and permeable substrate. The circuit substrate includes an electric circuit. A semiconductor chip is mounted on the circuit substrate. The semiconductor chip can receive a first electric signal from the temperature sensor in response to a temperature measurement of the user's skin. The semiconductor chip can receive a second electric signal from the electrodes in response to electric signals picked up from the user's body.

### BACKGROUND OF THE INVENTION

The present application relates to electronic devices, and in particular, to wearable patches that can attach to human skin for conducting measurement.

Electronic patches can be used for tracking objects and for performing functions such as producing sound, light or vibrations, and so on. As applications and human needs become more sophisticated and complex, electronic patches are required to perform a rapidly increasing number of tasks. Electronic patches are often required to be conformal to curved surfaces, which in the case of human body, can vary overtime.

Electronic patches can communicate with smart phones and other devices using WiFi, Bluetooth, Near Field Communication (NFC), and other wireless technologies. NFC is a wireless communication standard that enables two devices to quickly establish communication within a short range around radio frequency of 13.56 MHz. NFC is more secure than other wireless technologies such as Bluetooth and Wi-Fi because NFC requires two devices in close proximity (e.g. less than 10 cm). NFC can also lower cost comparing to other wireless technologies by allowing one of the two devices to be passive (a passive NFC tag).

Bluetooth is another wireless communication standard for exchanging data over relatively longer distances (in tens of meters). It employs short wavelength UHF radio waves from 2.4 to 2.485 GHz from fixed or mobile devices. Bluetooth devices have evolved to meet the increasing demand for low-power solutions that is required for wearable electronics. Benefited from relatively longer reading distance and active communication, Bluetooth technologies allow wearable patches to continuously monitoring vital information without human interference, which is an advantage over NFC in many applications.

Wearable patch (or tag) is an electronic patch to be worn by a user. A wearable patch is required to stay on user's skin and operate for an extended period of time from hours to months. A wearable patch can contain a micro-electronic system that can be accessed using NFC, Bluetooth, WiFi, or other wireless technologies. A wearable patch can be integrated with different sensors such as vital signs monitoring, motion track, skin temperature measurements, and ECG detection.

Despite recent development efforts, current wearable patches still suffer several drawbacks: they may not provide adequate comfort for users to wear them; they may not stay attached to user's body for the required length of time; and they are usually not aesthetically appealing. The conventional wearable patches also include rigid polymer substrates that are not very breathable. The build-up of sweat and moisture can cause discomfort and irritation to the skin, especially after wearing it for an extended period of time.

Conventional wearable thermometer patches have the additional challenge of inaccurate temperature measurement due to factors such as thermal resistance between the temperature sensor and the human skin, conduction loss of the temperature sensor to the ambient environment, as well as temperature reduction in the user skin caused by the thermal conduction to the wearable patch. Moreover, conventional wearable thermometer patches can also have slow measurement responses.

Another challenge is that it is extremely difficult to measure the surface temperature accurately, especially when measuring the human skin temperature which being impacted by the blood circulation under the skin. Several critical factors can impact the continuous measurement of armpit temperature: the ambient temperature can impact temperature measurement when arm is opened; and thermal contact resistance can change when the contact between the temperature sensing unit and human skin became loose.

Moreover, conventional wearable devices are often not robust enough to sustain repeated elongations during the movements of the body that the wearable patches are attached to. Under stress, different layers in wearable patches can break or delaminate rendering the patches inoperable. On the other hand, conventional electrodes for measuring human organ electrical activities are usually bulky and not stretchable or flexible. The data collection process is usually at the cost of life quality for users.

Another challenge for conventional wearable thermometer patches is that the user's skin may interfere with their proper wireless communications. For example, the antenna's communication range can be significantly reduced by the adjacency to user's skin. The wireless communication range of an antenna in contact with the skin is less than half the range for an antenna that is placed 4 mm away from the user's skin.

There is therefore a need for a flexible wearable electronic patch that can correctly measure temperatures of user's skin with high accuracy and fast response time, while capable of performing wireless communications in a required range.

### SUMMARY OF THE INVENTION

The presently disclosure attempts to address the aforementioned limitations in conventional electronic patches. The presently disclosed wearable wireless thermometer patch that can be attached to human skin to conduct temperature measurements with high accuracy and faster respond time.

Moreover, the disclosed wearable patches include flexible, stretchable, or conforming electrodes that are configured for measuring human organ electrical activities such as ECG, EEG, EMG, etc. The disclosed wearable patches are capable of measuring electrical signals in human body and are very stretchable, flexible, breathable, and comfortable to use.

In one general aspect, the present invention relates to a wearable patch which includes a stretchable and permeable substrate comprising a first opening; a temperature sensing unit mounted in the stretchable and permeable substrate, wherein the temperature sensing unit includes a temperature sensor configured to measure a user's skin temperature; one or more electrodes attached to the stretchable and permeable substrate, wherein the one or more electrodes comprise a bottom that can come to contact with the user's skin and to pick up electric signals from the user's body; a flexible circuit substrate on the stretchable and permeable substrate, wherein the flexible circuit substrate includes an electric circuit; and a semiconductor chip mounted on the flexible circuit substrate, wherein the temperature sensor, the one or more electrodes, and the semiconductor chip are electrically connected with the electric circuit in the flexible circuit substrate, wherein the semiconductor chip is configured to receive a first electric signal from the temperature sensor in response to a temperature measurement of the user's skin, wherein the semiconductor chip can receive a second electric signal from the one or more electrodes in response to electric signals picked up from the user's body. Implementations of the system may include one or more of the following. The wearable patch can further include an antenna mounted on the circuit substrate and in electric connection with the semiconductor chip, wherein the semiconductor chip can produce electric signals to enable the antenna to wirelessly exchange data with an external device, wherein the data include the temperature measurement and the electric signals picked up from the user's body. The circuit substrate can be folded to define an inner surface, wherein the circuit substrate includes a first portion on the stretchable and permeable substrate and a second portion away from the stretchable and permeable substrate, wherein the antenna is mounted on the inner surface of the circuit substrate. The wearable patch can further include a battery electrically connected to the electric circuit in the circuit substrate and configured to supply power to the semiconductor chip, wherein the battery is mounted on the stretchable and permeable substrate or the circuit substrate. The circuit substrate can be folded to define an inner surface, wherein the circuit substrate can include a first portion on the stretchable and permeable substrate and a second portion away from the stretchable and permeable substrate, wherein the semiconductor is mounted on the inner surface of the circuit substrate. The temperature sensing unit can include a thermally conductive cup having a bottom portion mounted in the first opening and fixed to the stretchable and permeable substrate, wherein the temperature sensor is positioned inside and is in thermal conduction with the thermally conductive cup. The temperature sensing unit can include a thermally-conductive adhesive that fixes the temperature sensor to an inner surface of the thermally conductive cup; and a thermally insulating material in a top portion of the thermally conductive cup. The one or more electrodes can include at least two electrodes configured to measure a voltage the user's body. The one or more electrodes can include conductive layers formed on a bottom surface of the stretchable and permeable substrate. The one or more electrodes can include a grid of electrically conducting wires. The one or more electrodes can include an electrically conductive cup that is electrically connected to the electric circuit in the second circuit substrate, wherein the stretchable and permeable substrate comprising a second opening in which the electrically conductive cup is mounted. The wearable patch can further include a plastic layer in between the stretchable and permeable substrate and the circuit substrate, wherein the plastic layer forms a portion of the electrically conductive cup in the one or more electrodes; and a layer of a conductive material coated on the lower surface of the plastic layer and in electric connection with the electric circuit in the circuit substrate, wherein the layer of the conductive material forms a portion of the electrically conductive cup in the one or more electrodes. The electrically conductive cup can provide a spring load under pressure, which presses a bottom surface of the electrically conductive cup to contact to the user's skin. The one or more electrodes can include two electrically conductive cups that are respectively electrically connected to the electric circuit in the second circuit substrate, wherein the stretchable and permeable substrate can include a second opening and a third opening in which the electrically conductive cups are respectively mounted. The wearable patch can further include an adhesive layer between the stretchable and permeable substrate and the circuit substrate. The wearable patch can further include an elastic layer formed on the stretchable and permeable substrate, the circuit substrate, and the temperature sensing unit. The wearable patch can further include a plastic layer in on the stretchable and permeable substrate and below the circuit substrate and the elastic layer; and a channel is formed between the stretchable and permeable substrate and the plastic layer configured to vent moisture from the user's skin. The stretchable and permeable substrate can be formed by a foam material.

These and other aspects, their implementations and other features are described in detail in the drawings, the description and the claims.

### BRIEF DESCRIPTION OF THE DRAWINGS

FIG. 1 illustrates a wearable patch attached to a user's skin.

FIG. 2 is a cross-sectional view of a wearable patch for measuring temperature and electrical signals in accordance with some embodiments of the present invention.

FIG. 3 is a detailed cross-sectional view of an exemplified temperature sensing unit in the wearable patch in accordance with some embodiments of the present invention.

FIG. 4 is a cross-sectional view of another wearable patch for measuring temperature and electrical signals in accordance with some embodiments of the present invention.

FIG. 5 is a cross-sectional view of another wearable patch for measuring temperature and electrical signals in accordance with some embodiments of the present invention.

### DETAILED DESCRIPTION OF THE INVENTION

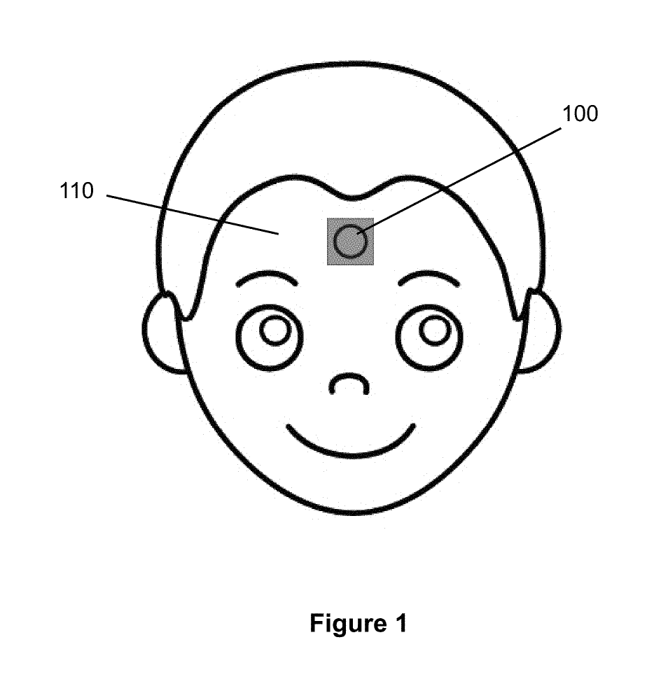

Referring to FIG. 1, a wearable patch 100 is attached to a user's skin 110 for measuring body vital signs. The wearable patch 100 can be placed on forehead, hand, wrist, arm, shoulder, waist, leg, foot, armpit, or other parts of the body. In the present disclosure, the term “wearable patch” can also be referred to as “wearable sticker” or “wearable tag”.

As discussed above, wearable electronic patches face several challenges: the user's skin 110 may interfere with their proper operations. For example, the wearable patch 100 may include an antenna for wireless communications with other devices. The antenna's communication range can be significantly reduced when an antenna is placed in contact with the user's skin 110.

The presently disclosure aims to overcome the drawbacks in conventional wearable patches, and to provide highly stretchable, compliant, durable, breathable, and comfortable wearable electronic patches while performing more accurate and more responsive measurements and communication functions.

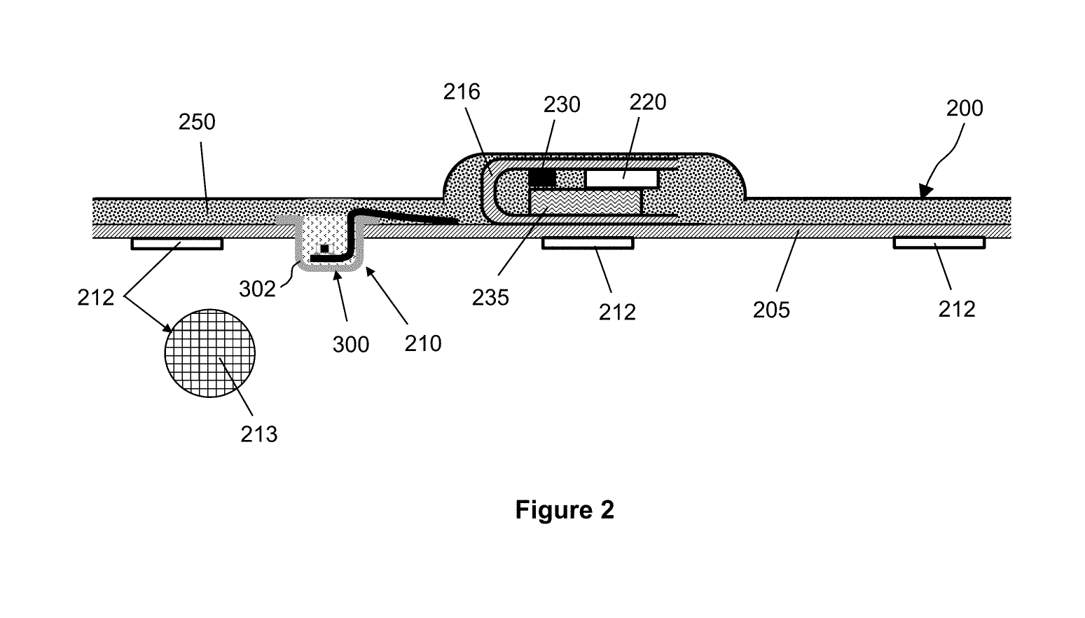

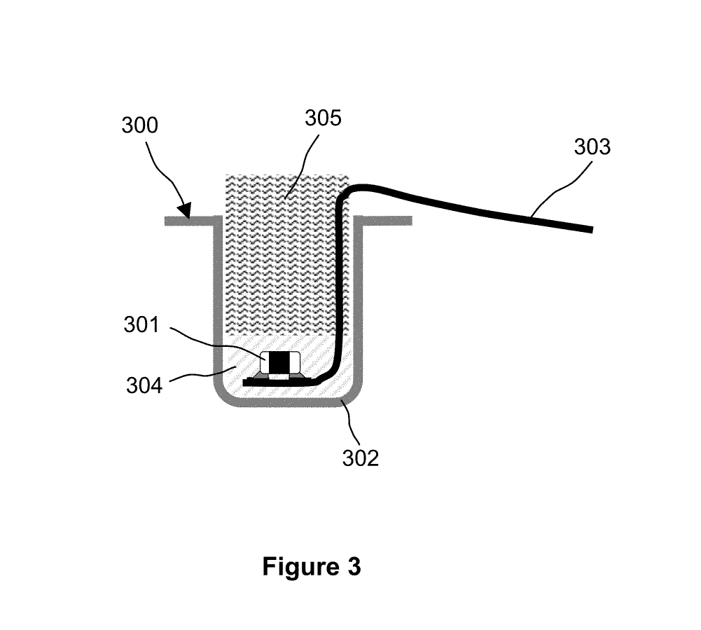

Referring to FIGS. 2 and 3, a wearable patch 200 includes a stretchable and permeable substrate 205 having a first electric circuit (not shown) embedded in or formed on. The stretchable and permeable substrate 205 can be made of soft foam materials such as EVA, PE, CR, PORON, EPD, SCF or fabric textile, to provide stretchability and breathability. The stretchable and permeable substrate 205 has a large opening 210 in which a temperature sensing unit 300 in mounted. One or more electrodes 212 include conductive layers formed on a bottom surface of the stretchable and permeable substrate 205 and in electric connection with the first electric circuit. The one or more electrodes 212 are formed by a flexible material or in a flexible structure adapted to provide conforming electrical contacts to a user's skin to collect the ECG signals. For example, each electrode 212 can be made of a grid of electrically conducting wires 213.

A folded flexible circuit substrate 216 is formed on the stretchable and permeable substrate 205. The folded flexible circuit substrate 216 includes a second electric circuit (not shown) embedded in or formed on that is electrically connected with the first electric circuit. The folded flexible circuit substrate 216 and the stretchable and permeable substrate 205. The folded flexible circuit substrate 216 includes a fold that defines an inner surface. The folded flexible circuit substrate 216 includes a first portion on the stretchable and permeable substrate 205 and a second portion away from the stretchable and permeable substrate 205.

A semiconductor chip 220, an antenna 230, and a battery 235 are mounted or formed on the inner surface of the folded flexible circuit substrate 216. The semiconductor chip 220, the antenna 230, and the battery 235 are connected with the second electric circuit in the folded flexible circuit substrate 216. The semiconductor chip 220, the antenna 230, and the battery 235 can be formed an inside surface of the folded flexible circuit substrate 216, which provides physical protection to these components.

An elastic layer 250 is formed on the folded flexible circuit substrate 216, the stretchable and permeable substrate 205 and the temperature sensing unit 300. The elastic layer 250 can be bonded to the folded flexible circuit substrate 216 and the stretchable and permeable substrate 205 with an adhesive material. The elastic layer 250 can also be directly formed on them by molding without using a bonding material. In the latter case, the elastic layer 250 can be formed a stretchable and breathable material such as with liquid silicone.

The elastic layer 250 can include one or more cavities for enhancing flexibility (bendable) and stretchability of the elastic layer 250 and the whole wearable patch 200. The elastic layer 250 can be made of a non-conductive material such as an elastomeric material or a viscoelastic polymeric material having low Young's modulus and high failure strain. In some embodiments, the elastic layer 250 has a Young's Modulus <0.3 Gpa. In some cases, the elastic layer 250 and can have Young's Modulus <0.1 Gpa to provide enhanced flexibility and tackability. Materials suitable for the elastic layer 250 include elastomers, viscoelastic polymers, such as silicone, porous foam, and medical grade polyurethane that is a transparent medical dressing used to cover and protect wounds with breathability and conformation to skin.

In usage, an adhesive material formed on the lower surface of the stretchable and permeable substrate 205 is attached the user's skin, so that the bottom of the thermally conductive cup 302 and electrodes 212 are in tight contact with a user's skin to accurately measure temperature and electric signals from the user's skin. The semiconductor chip 220 receives a first electric signal from the temperature sensor 301 in response to a temperature measurement of the user's skin. The semiconductor chip 220 can also receive a second electric signal from the electrodes 212 in response to electric signals picked up from the user's body. When the wearable patch 200 is worn by a user, the antenna 230 is separated from the user's skin by the folded flexible circuit substrate 216 and the stretchable and permeable substrate 205, which minimizes the impact of the user's body on the transmissions of wireless signals by the antenna 230.

Further details of wearable thermometer patches are disclosed in the commonly assigned co-pending U.S. patent application Ser. No. 14/814,347 “Three dimensional electronic patch”, filed Jul. 30, 2015, the disclosure of which is incorporated herein by reference.

The temperature sensing unit 300 includes a thermally conductive cup 302 having its bottom portion mounted into the large opening 210 and fixed to the stretchable and permeable substrate 205 by an adhesive. The bottom portion of the thermally conductive cup 302 protrudes out of the lower surface of the stretchable and permeable substrate 205. The lips of the thermally conductive cup 302 near its top portion are fixedly attached or bonded to bonding pads (not shown) on the stretchable and permeable substrate 205 by soldering or with an adhesive. The thermally conductive cup 302 can be made of a thermally conductive metallic or alloy material such as copper, stainless steel, ceramic or carbide composite materials.

A temperature sensor 301 is attached to and in thermal conduction with an inner surface near the bottom of the thermally conductive cup 302. The temperature sensor 301 can be implemented, for example, by a thermistor, a Resistor Temperature Detector, or a Thermocouple. When an outer surface of the bottom portion of the thermally conductive cup 302 is in contact with a user's skin, the thermally conductive cup 302 can thus effectively transfer heat from a user's skin to the temperature sensor 301. A flexible conductive ribbon 303 is connected to the temperature sensor 301 in the thermally conductive cup 302 and to the first electric circuit in the stretchable and permeable substrate 205. Thus the temperature sensor 301 can send an electric signal to the semiconductor chip 220 via the first electric circuit and the second electric circuit in response to a measured temperature. The semiconductor chip 220 processes the electric signal and output another electrical signal which enables the antenna 230 to transmit a wireless signal carrying the measurement data to another external device such as a mobile phone or a computer. The wireless signal can be based on using WiFi, Bluetooth, Near Field Communication (NFC), and other wireless standards. The battery 235 powers the semiconductor chip 220, the antenna 230, the first and the second electric circuits, and possibly the temperature sensor 301.

The temperature sensor 301 can be fixed to an inner surface at the bottom of the thermally conductive cup 302 by a thermally-conductive adhesive 304, which allows effective heat transfer from the bottom of the thermally conductive cup 302 to the temperature sensor 301. Examples of the thermally-conductive adhesive 304 can include electrically-insulative thermally-conductive epoxies and polymers. A thermally insulating material 305 filling the top portion of the thermally conductive cup 302 fixes the thermally-conductive adhesive 304 at the bottom of the thermally conductive cup 302 and reduces heat loss from the temperature sensor 301 to the elastic layer (described below) or the environment. The flexible conductive ribbon 303 can be bent and laid out along the wall the thermally conductive cup 302.

Further details of the temperature sensing unit are disclosed in the commonly assigned co-pending U.S. patent application Ser. No. 15/224,121 “Wearable thermometer patch for accurate measurement of human skin temperature”, filed Jul. 29, 2016, and commonly assigned co-pending U.S. patent application Ser. No. 15/406,380 “A wearable thermometer patch for correct measurement of human skin temperature”, filed Jan. 13, 2017 the disclosure of which is incorporated herein by reference.

The disclosed wearable thermometer patch can significantly enhance measurement accuracy and responsiveness, and reduce thermal noise. The temperature sensing unit 300 is positioned in contact with a user's skin. The temperature sensor is placed at the bottom of a thermally conductive cup and in good thermal conduction with the user's skin. The minimized thermal resistance between the temperature sensor and the user's skin reduces temperature measurement error and also decreases measurement response time. Moreover, the temperature sensor is secured fixed by an adhesive to the bottom of the thermally conductive cup such that the temperature sensor is not affected and detached by user's body movements, which improves durability of the wearable thermometer patch. Furthermore, the temperature sensor is thermally isolated with the ambient environment by a thermal insulating material in the top portion of the thermally conductive cup. The reduced thermal capacity helps further reduces background noise in the measurements of user's skin temperature and increase response rate of measurement. A layer of soft perforated polymer material under the flexible substrate minimizes heat conduction from the user's skin to the wearable thermometer patch, thus reducing the “cooling effect” of the user's skin by the wearable thermometer patch.

Another advantage of the disclosed wearable thermometer patch is that it is stretchable, compliant, durable, and comfortable to wear by users. The disclosed wearable thermometer patch includes a flexible substrate covered and protected by an elastic layer that increases the flexibility and stretchability.

Yet another advantage of the disclosed wearable thermometer patch is that it can significantly increase wireless communication range by placing the antenna on the upper surface of the flexible circuit substrate. The thickness of the substrate as well as the height of the thermally conductive cup can be selected to allow enough distance between the antenna and the user's skin to minimize interference of user's body to the wireless transmission signals.

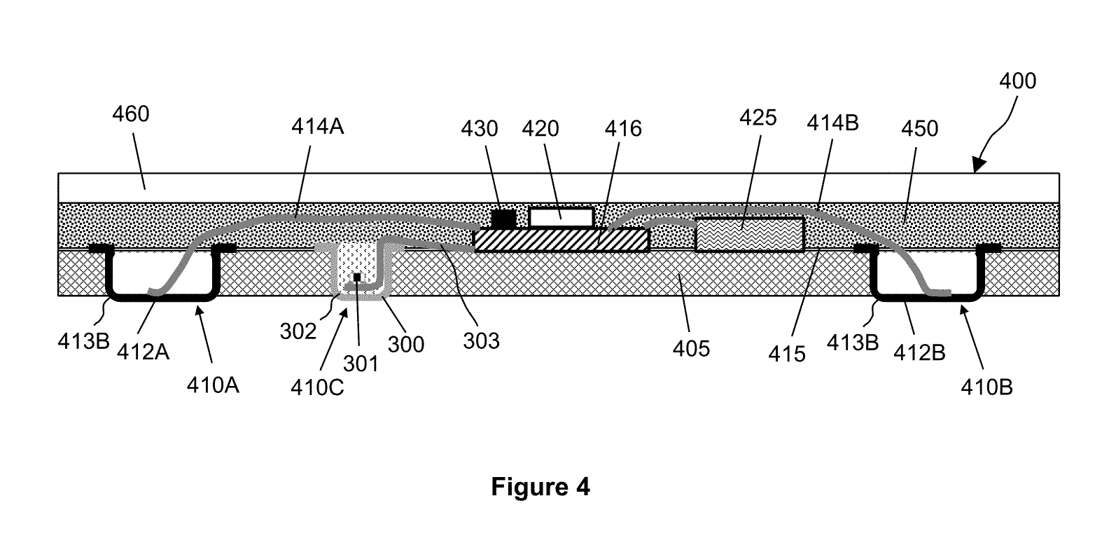

In some embodiments, referring to FIG. 4, a wearable patch 400 includes a stretchable and permeable substrate 405 that include openings 410A, 410B, 410C. The stretchable and permeable substrate 405 can be made of soft foam materials such as EVA, PE, CR, PORON, EPD, SCF or fabric textile, to provide stretchability and breathability. A temperature sensing unit 300, with details shown in FIG. 3, is mounted in the opening 410C. Two electrodes 412A, 412B, respectively formed by electrically conductive cups 413A, 413B, are mounted in the openings 410A, 410B. A circuit substrate 416 and a battery 425 are bonded to the stretchable and permeable substrate 405 by an adhesive layer 415 pre-laminated on the stretchable and permeable substrate 405. A semiconductor chip 420 and an antenna 430 are mounted on the circuit substrate 416. The circuit substrate 416 includes an electric circuit therein and can for example be implemented with a printed circuit board.

The temperature sensing unit 300, as described above, includes a thermally conductive cup 302 having its bottom portion mounted into the large opening 410C and fixed to the stretchable and permeable substrate 405 by an adhesive. A temperature sensor 301 is electrically connected to the electric circuit in the circuit substrate 416 by a flexible conductive ribbon 303.

The electrically conductive cups 413A, 413B in the electrodes 412A, 412B are respectively electrically connected to the electric circuit in the circuit substrate 416 by conductive lines 414A, 414B (e.g. flexible ribbons embedded with conductive circuits). When the wearable patch 400 is attached to a user, the bottom surfaces of the electrically conductive cups 413A, 413B are configured to come into contact with the user's skin to pick up electric signals from the user's body. A voltage can be measured between the electrodes 412A, 412B.

An elastic layer 450 is formed on the stretchable and permeable substrate 405, the circuit substrate 416, the temperature sensing unit 300, and the electrodes 412A, 412B. The elastic layer 450 can be formed by soft stretchable foam and permeable materials such as EVA, PE, CR, PORON, EPD, SCF, or fabric textile. A thin film 460 is formed on the elastic layer 450 for protection and cosmetic purposes.

In usage, an adhesive material formed on the lower surface of the stretchable and permeable substrate 405 is attached the user's skin, so that the bottom of the thermally conductive cup 302 and the bottom surfaces of the electrodes 412A, 412B are in tight contact with a user's skin to accurately measure temperature and electric signals from the user's skin. The semiconductor chip 420 receives a first electric signal from the temperature sensor 301 in response to a temperature measurement of the user's skin. The semiconductor chip 420 can also receive a second electric signal from the electrodes 412A, 412B in response to electric signals picked up from the user's body.

The semiconductor chip 420 processes the electric signal and output another electrical signal which enables the antenna 430 to transmit a wireless signal carrying the measurement data to another external device such as a mobile phone or a computer. The wireless signal can be based on using WiFi, Bluetooth, Near Field Communication (NFC), and other wireless standards. When the wearable patch 400 is worn by a user, the antenna 430 is separated from the user's skin by the circuit substrate 416 and the stretchable and permeable substrate 405, which minimizes the impact of the user's body on the transmissions of wireless signals by the antenna 430.

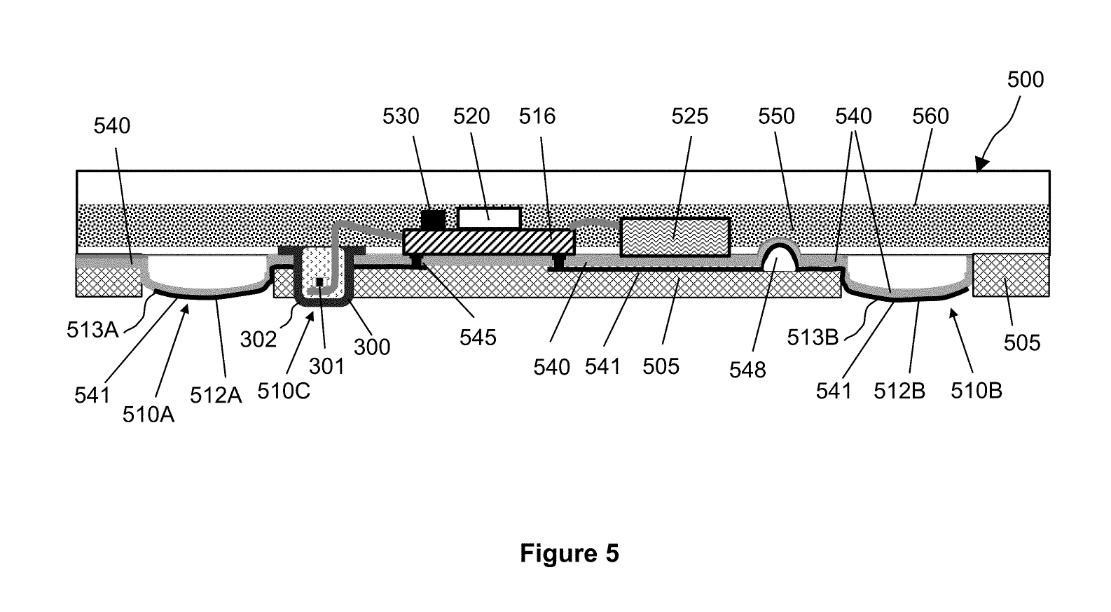

In some embodiments, referring to FIG. 5, a wearable patch 500 includes a stretchable and permeable substrate 505 that include openings 510A, 510B, 510C. The stretchable and permeable substrate 505 can be made of soft foam materials such as EVA, PE, CR, PORON, EPD, SCF or fabric textile, to provide stretchability and breathability. A plastic layer 540 is formed on the stretchable and permeable substrate 505, and in the openings of 510A and 519B. The plastic layer 540 can for example be made of a PET film, TPU film, or a fabric. A thin layer 541 of an electrically-conductive material is coated or printed on the lower surface of the plastic layer 540. Examples suitable for the electrically-conductive material include AgCl, a carbon paste, or metal-polymer paste.

The portions of the plastic layer 540 and the associated portion of the thin layer 541 underneath in the openings of 510A and 519B respectively form electrically conductive buttons 513A, 513B, which respectively form electrodes 512A, 512B in the openings 510A, 510B. In usage, the electrically conductive buttons 513A, 513B function as electrodes for picking electrical signals from the user's skin (e.g. in ECG measurements). The electrically conductive buttons 513A, 513B can sustain spring loads, which, when under a slight pressure, presses the bottom surface of the electrically conductive buttons 513A, 513B form a good contact with the user's skin.

A circuit substrate 516 and a battery 525 are bonded to the stretchable and permeable substrate 505 by an adhesive layer 515 pre-laminated on the stretchable and permeable substrate 505. A semiconductor chip 520 and an antenna 530 are mounted on the circuit substrate 516. The circuit substrate 516 includes an electric circuit therein and can for example be implemented with a printed circuit board. The thin layer 541 of the electrically-conductive material is electrically connected to the electric circuit in the circuit substrate 516 through via 545.

A temperature sensing unit 300, with details shown in FIG. 3, is mounted in the opening 510C. The temperature sensing unit 300 includes a thermally conductive cup 302 having its bottom portion mounted into the large opening 510C and fixed to the stretchable and permeable substrate 505 and the plastic layer 540 thereon by an adhesive. A temperature sensor 301 is electrically connected to the electric circuit in the circuit substrate 516 by a flexible conductive ribbon 303.

The electrically conductive buttons 513A, 513B in the electrodes 512A, 512B are respectively electrically connected to the electric circuit in the circuit substrate 516 by the thin layer 541 of the electrically-conductive material and via 545. When the wearable patch 500 is attached to a user, the portion of the thin layer 541 of the electrically-conductive material at the bottom surfaces of the electrically conductive buttons 513A, 513B come into contact with the user's skin to pick up electric signals from the user's body. A voltage can be measured between the electrodes 512A, 512B.

An elastic layer 550 is formed on the plastic layer 540 on the stretchable and permeable substrate 505, the circuit substrate 516, the temperature sensing unit 300, the electrodes 512A, 512B, and other electronic components. The elastic layer 550 can be formed by soft stretchable foam materials such as EVA, PE, CR, PORON, EPD, SCF, or fabric textile. A thin film 560 is formed on the elastic layer 550 for protection and cosmetic purposes.

In usage, an adhesive material formed on the lower surface of the stretchable and permeable substrate 505 is attached the user's skin, so that the bottom of the thermally conductive cup 302 and the bottom surfaces of the electrodes 512A, 512B are in tight contact with a user's skin to accurately measure temperature and electric signals from the user's skin. The semiconductor chip 520 receives a first electric signal from the temperature sensor 301 in response to a temperature measurement of the user's skin. The semiconductor chip 520 can also receive a second electric signal from the electrodes 512 in response to electric signals picked up from the user's body through the thin layer 541 of the electrically-conductive material and via 545.

The semiconductor chip 520 processes the electric signal and output another electrical signal which enables the antenna 530 to transmit a wireless signal carrying the measurement data to another external device such as a mobile phone or a computer. The wireless signal can be based on using WiFi, Bluetooth, Near Field Communication (NFC), and other wireless standards. When the wearable patch 500 is worn by a user, the antenna 530 is separated from the user's skin by the circuit substrate 516 and the stretchable and permeable substrate 505, which minimizes the impact of the user's body on the transmissions of wireless signals by the antenna 530.

A portion of the plastic layer 540 and the associated portion of the thin layer 541 form a channel 548 between the elastic layer and the stretchable and permeable substrate 505. The channel 548 has a dome shape and defines an air passage for venting moisture from the user's skin to the ambient environment.

The above disclosed wearable patches can make monitoring system more compact and more effective. The dual measurements of temperature and EEG signals can have several advantages. For example, in monitoring the human bio-signals, the heart beat variations extracted from the EEG signal accompanying skin temperature not only can provide more useful information for diagnosis, but also can provide more accurate information for health such as sleep quality. Doctors can make better judgments of patients' health condition by simultaneous monitoring the patients' human bio-signals.

The disclosed wearable thermometer patches can also include electronic components such as the semiconductor chips, resistors, capacitors, inductors, diodes (including for example photo sensitive and light emitting types), other types of sensors, transistors, amplifiers. The sensors can also measure temperature, acceleration and movements, and chemical or biological substances. The electronic components can also include electromechanical actuators, chemical injectors, etc. The semiconductor chips can perform communications, logic, signal or data processing, control, calibration, status report, diagnostics, and other functions.

While this document contains many specifics, these should not be construed as limitations on the scope of an invention that is claimed or of what may be claimed, but rather as descriptions of features specific to particular embodiments. Certain features that are described in this document in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination.

Only a few examples and implementations are described. Other implementations, variations, modifications and enhancements to the described examples and implementations may be made without deviating from the spirit of the present invention.

What is claimed is:

1. A wearable patch, comprising:
a stretchable and permeable substrate comprising a first opening;
a temperature sensing unit mounted in the stretchable and permeable substrate, wherein the temperature sensing unit comprises a temperature sensor configured to measure a user's skin temperature;
one or more electrodes attached to the stretchable and permeable substrate, wherein the one or more electrodes comprise a bottom surface configured to come to contact with the user's skin and to pick up electric signals from the user's body;
a circuit substrate on the stretchable and permeable substrate, wherein the circuit substrate comprises an electric circuit; and
a semiconductor chip mounted on the circuit substrate, wherein the temperature sensor, the one or more electrodes, and the semiconductor chip are electrically connected with the electric circuit in the circuit substrate, wherein the semiconductor chip is configured to receive a first electric signal from the temperature sensor in response to a temperature measurement of the user's skin, wherein the semiconductor chip is configured to receive a second electric signal from the one or more electrodes in response to electric signals picked up from the user's body.

2. The wearable patch of claim 1, further comprising:
an antenna mounted on the circuit substrate and in electric connection with the semiconductor chip, wherein the semiconductor chip is configured to produce electric signals to enable the antenna to wirelessly exchange data with an external device, wherein the data include the temperature measurement and the electric signals picked up from the user's body.

3. The wearable patch of claim 2, wherein the circuit substrate is folded to define an inner surface, wherein the circuit substrate includes a first portion on the stretchable and permeable substrate and a second portion away from the stretchable and permeable substrate, wherein the antenna is mounted on the inner surface of the circuit substrate.

4. The wearable patch of claim 1, further comprising:
a battery electrically connected to the electric circuit in the circuit substrate and configured to supply power to the semiconductor chip, wherein the battery is mounted on the stretchable and permeable substrate or the circuit substrate.

5. The wearable patch of claim 1, wherein the circuit substrate is folded to define an inner surface, wherein the circuit substrate includes a first portion on the stretchable and permeable substrate and a second portion away from the stretchable and permeable substrate, wherein the semiconductor is mounted on the inner surface of the circuit substrate.

6. The wearable patch of claim 1, wherein the temperature sensing unit includes a thermally conductive cup having a bottom portion mounted in the first opening and fixed to the stretchable and permeable substrate, wherein the temperature sensor is positioned inside and is in thermal conduction with the thermally conductive cup.

7. The wearable patch of claim 6, wherein the temperature sensing unit comprises:
a thermally-conductive adhesive that fixes the temperature sensor to an inner surface of the thermally conductive cup; and
a thermally insulating material in a top portion of the thermally conductive cup.

8. The wearable patch of claim 1, wherein the one or more electrodes include at least two electrodes configured to measure a voltage the user's body.

9. The wearable patch of claim 1, wherein the one or more electrodes include conductive layers formed on a bottom surface of the stretchable and permeable substrate.

10. The wearable patch of claim 9, wherein the one or more electrodes include a grid of electrically conducting wires.

11. The wearable patch of claim 1, wherein the one or more electrodes include an electrically conductive cup that is electrically connected to the electric circuit in the second circuit substrate, wherein the stretchable and permeable substrate comprises a second opening in which the electrically conductive cup is mounted.

12. The wearable patch of claim 11, further comprising:
a plastic layer in between the stretchable and permeable substrate and the circuit substrate, wherein the plastic layer forms a portion of the electrically conductive cup in the one or more electrodes; and
a layer of a conductive material coated on the lower surface of the plastic layer and in electric connection with the electric circuit in the circuit substrate, wherein the layer of the conductive material forms a portion of the electrically conductive cup in the one or more electrodes.

13. The wearable patch of claim 11, wherein the electrically conductive cup is configured to provide a spring load under pressure, which presses a bottom surface of the electrically conductive cup to contact to the user's skin.

14. The wearable patch of claim 1, wherein the one or more electrodes include two electrically conductive cups that are respectively electrically connected to the electric circuit in the second circuit substrate, wherein the stretchable and permeable substrate comprising a second opening and a third opening in which the electrically conductive cups are respectively mounted.

15. The wearable patch of claim 1, further comprising:
an adhesive layer between the stretchable and permeable substrate and the circuit substrate.

16. The wearable patch of claim 1, further comprising:
an elastic layer formed on the stretchable and permeable substrate, the circuit substrate, and the temperature sensing unit.

17. The wearable patch of claim 16, further comprising:
a plastic layer in on the stretchable and permeable substrate and below the circuit substrate and the elastic layer; and
a channel is formed between the stretchable and permeable substrate and the plastic layer configured to vent moisture from the user's skin.

18. The wearable patch of claim 1, wherein the stretchable and permeable substrate is formed by a foam material.
